(19) United States
(12) Patent Application Publication
(10) Pub. No.: US 2018/0207036 A1
(43) Pub. Date: Jul. 26, 2018

(54) TAMPON

(71) Applicant: Yuan-Cheng CHIEN, Kaohsiung City (TW)

(72) Inventor: Yuan-Cheng CHIEN, Kaohsiung City (TW)

(21) Appl. No.: 15/469,058

(22) Filed: Mar. 24, 2017

(30) Foreign Application Priority Data
Jan. 25, 2017 (TW) 106102776

(51) Int. Cl. A61F 13/20; A61F 13/34; A61F 13/26

(52) U.S. Cl. CPC A61F 13/2022; A61F 13/202; A61F 13/26; A61F 13/34; A61F 13/2051

(57) ABSTRACT

A tampon includes an absorbent body and a string, the absorbent body has a helical portion and a cushion space, the cushion space is axially formed in the helical portion, and the string is disposed on one end of the absorbent body.

### CROSS-REFERENCE TO RELATED APPLICATION

Pursuant to 35 U.S.C. § 119(a), this application claims the benefit of the priority to Taiwan Patent Application No. 106102776 filed Jan. 25, 2017. The content of the prior application is incorporated herein by its entirety.

### BACKGROUND OF THE INVENTION

### 1. Field of the Invention

The present invention relates to feminine hygiene articles, particularly a tampon.

### 2. Description of the Prior Arts

A tampon is a solid cylinder of cotton which can absorb the menstrual blood from female endometrial exfoliation and is placed at the vaginal orifice to prevent menstrual blood leakage, so that the user can do any activities or exercise as usual during menstruation. The tampon is more convenient compared to a sanitary napkin and thus is gradually accepted and widely used.

However, after use for a period of time, the tampon will expand due to menstrual blood absorption, which may make the user feel uncomfortable. Furthermore, the increasing volume of the tampon may increase the difficulty of removing the tampon, and may injure the user during the removal process. Therefore, the conventional tampons still have room for improvement.

### SUMMARY OF THE INVENTION

In view of the problems above-mentioned, the object of the present invention is to reduce the swelling of the tampon after menstrual blood absorption to mitigate uncomfortable feeling during use, and to reduce the difficulty of removing the tampon, so that convenience in use of the tampon is improved and the tampon is more widely accepted and used.

Accordingly, a tampon of the present invention comprises an absorbent body, which has a helical portion and a cushion space, the cushion space is axially formed in the helical portion, and a string is disposed on one end of the absorbent body.

By means of the above technical means, the tampon of the present invention is not solid but has a cushion space in the absorbent body as a cushion area for the volume change of the absorbent body. Moreover, the dispositions of the cushion space and the helical portion make the absorbent body swell only slightly without drastic enlargement in volume, so that the user can easily remove the tampon without any discomfort. Compared to conventional solid tampons, the tampon of the present invention has improved convenience in use and reduces the risk of injury during removal of the absorbent body. Even when the absorbent body has a relatively large expansion due to the absorption of a large amount of menstrual blood, the user can take the absorbent body out by stretching the string to straighten the helical portion to reduce the diameter of the absorbent body so that the convenience of withdrawing the absorbent body is enhanced. And the scallop pattern of the helical portion increases the air permeability of the absorbent body during use. Therefore, the tampon of the present invention provides improved comfort, air permeability, and convenience of use compared to the conventional tampon.

In addition, the absorbent body will be slightly expanded after absorbing the fluid, and the multiple helical rings of the helical portion slightly loosen, so that the absorbent body of the present invention has a larger absorption surface area, which can absorb blood more quickly. Even with instantaneous heavy flow of menstrual blood, the blood is absorbed immediately and will not leak onto the pants.

Preferably, the helical portion has multiple helical rings and each two adjacent ones of the multiple helical rings are closely abutted on each other.

Preferably, the absorbent body has a double thread segment and a single thread segment interconnected, the double thread segment has a concentric double helix, and the single thread segment has a single helix. Alternatively, the absorbent body has a double thread segment and two single thread segments interconnected, the double thread segment is disposed between the two single thread segments, the double thread segment has a concentric double helix, and the two single thread segments each have a single helix. The double thread segment of the absorbent body has a larger diameter than that of the single thread segment to prevent the absorbent body falling off from the vagina, and the single thread segment has the smaller diameter to place the absorbent body easily.

More preferably, the length of the double thread segment is half of the length of the single thread segment.

Preferably, the absorbent body is formed by twining an absorbent fiber, and the absorbent fiber is twined to form the helical portion. In addition, the absorbent fiber is preferably made of high density cotton fibers.

Preferably, the absorbent fiber has a shorter-diameter part, which is adjacent to the end of the helical portion that is opposite to the string. Thus, the absorbent body has the shorter-diameter part.

Preferably, the absorbent fiber is formed by multiple fiber monomers entwined.

Preferably, the cross section of the absorbent fiber is circular.

Preferably, both ends of the string are disposed on the absorbent body.

Preferably, the shape of the absorbent body is cylindrical, and the absorbent body has a larger-diameter end and a smaller-diameter end opposite each other, and the string is disposed on the larger-diameter end.

Other objectives, advantages and novel features of the invention will become more apparent from the following detailed description when taken in conjunction with the accompanying drawings.

### BRIEF DESCRIPTION OF THE DRAWINGS

FIG. 1 is a perspective view of the first embodiment of the tampon of the present invention;

FIG. 2 is a side cross-sectional view of the first embodiment of the tampon of the present invention in combination with an applicator;

FIG. 3 is a perspective view of the first embodiment of the tampon of the present invention in combination with the applicator;

FIG. 4 is a schematic view of the first step in the use of the first embodiment of the tampon of the present invention;

FIG. 5 is a schematic view of the second step in the use of the first embodiment of the tampon of the present invention;

FIG. 6 is a schematic view of the third step in the use of the first embodiment of the tampon of the present invention.

FIG. 7 is a schematic view of the fourth step in the use of the first embodiment of the tampon of the present invention;

FIG. 8 is a perspective view of the absorbent fiber of the second embodiment of the tampon of the present invention;

FIG. 9 is a perspective view of the third embodiment of the tampon of the present invention in combination with the applicator; and

FIG. 10 is a side cross-sectional view of the third embodiment of the tampon of the present invention in combination with the applicator.

### DETAILED DESCRIPTION OF THE PREFERRED EMBODIMENTS

Before the present invention is described in greater detail with reference to the accompanying embodiment, it should be noted herein that like elements are denoted by the same reference numerals throughout the present invention.

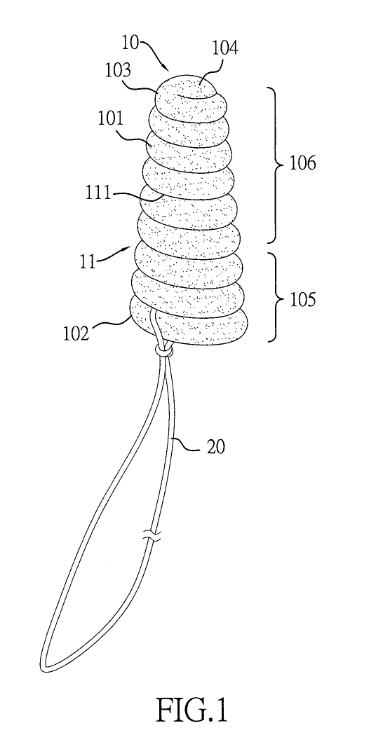

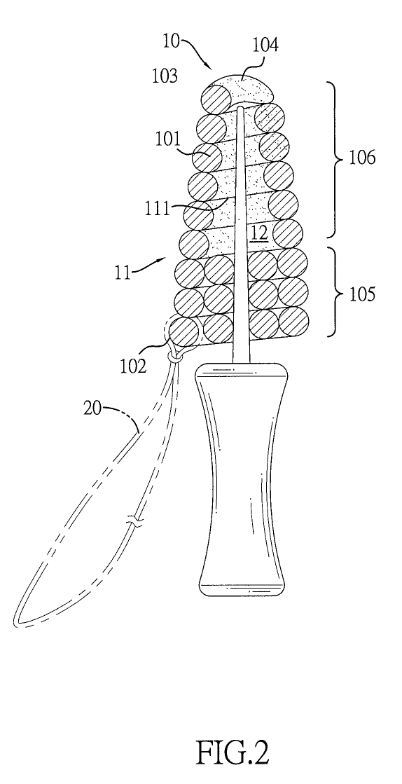

Referring to FIGS. 1 and 2, the first embodiment of the tampon in the present invention includes an absorbent body 10 and a string 20.

The absorbent body 10 is formed by twining an absorbent fiber 101, and the absorbent fiber 101 is made of cotton. The cross section of the absorbent fiber 101 is circular, and its diameter is not constant. The absorbent fiber 101 has a shorter-diameter part 104. The shape of the absorbent body 10 is cylindrical after the absorbent fiber 101 is twined, and the absorbent body 10, which is cylindrical, has a larger-diameter end 102 and a smaller-diameter end 103 opposite each other. In particular, the smaller-diameter end 103 is formed by twining the shorter-diameter part 104 of the absorbent fiber 101. The absorbent body 10 has a helical portion 11 and a cushion space 12.

The helical portion 11 is formed by the absorbent fibers 101 tightly entwined and abutted on each other spirally, has multiple helical rings 111, and each two of the multiple helical rings 111 adjacent to each other are closely abutted in the axial direction of the helical portion 11. That is, the distance between each two of the multiple helical rings 111 adjacent to each other is approximately equal to the diameter of the absorbing fibers 101, thereby forming the shape of the absorbent body 10.

Furthermore, as the absorbent body 10 has the multiple helical rings 111, the absorbent body 10 has a repeating scallop pattern in the appearance, and when in use, the protruding portion of the scallop pattern of the absorbent body 10 is attached to the vaginal wall and the recessed portion of the scallop pattern of the absorbent body 10, thereby forming an air-permeation space that increases the air permeability of the absorbent body 10 in use. Moreover, the multiple helical rings 111 adjacent to the larger-diameter end 102 are closely abutted on each other in the radial direction of the helical portion 11. In other words, several of the multiple helical rings 111 are concentrically arranged to form double helix rings, and since the double helix rings are each formed by two of the absorbent fibers 101 entwined, the larger-diameter ends 102 are formed. In another embodiment, the multiple helical rings 111 may be concentrically arranged to form multi-helix rings.

Specifically, the way of forming the helical portion 11 is folding the absorbent fiber 101 at a quarter of the full length thereof to form a folding end, while the absorbent fiber 101 has a double thread segment 105 and a single thread segment 106 interconnected, and the length of the double thread segment 105 is half of the length of the single thread segment 106. Then the double thread segment 105 is twined at the folding end so that the double thread segment 105 has a thickness of two absorbent fibers 101 thereof, hence the larger-diameter end 102 and the double helix rings arranged concentrically are formed after entwined. When twined spirally on the single thread segment 106, there is only one absorbent fiber 101, thus forming the smaller-diameter end 103. Finally, the absorbent body 10 is formed after the absorbent fiber 101 is formed in shape.

The cushion space 12 is axially formed in the helical portion 11. More specifically, the cushion space 12 is encircled by the multiple helical rings 111 of the helical portion 11.

The string 20 is disposed on one end of the absorbent body 10; in particular, both ends of the string 20 are disposed on the larger-diameter end 102 of the absorbent body 10, and thus the string 20 forms a loop to provide the user with ease in removal of the absorbent body 10 by pulling the string 20.

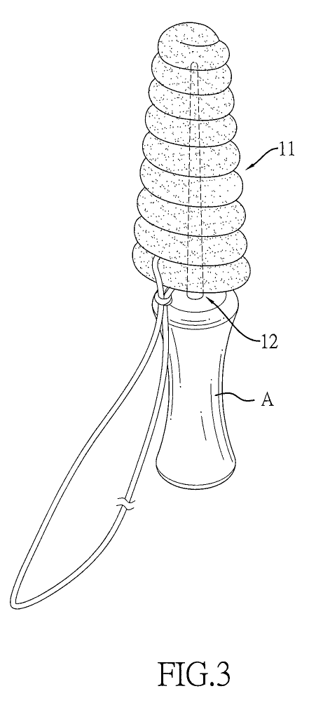

Referring to FIG. 3, since the cushion space 12 is formed in the helical portion 11, the tampon in the present invention can be combined with an applicator A which can be placed in the cushion space 12 to allow the user to put the tampon in the correct position with the applicator A.

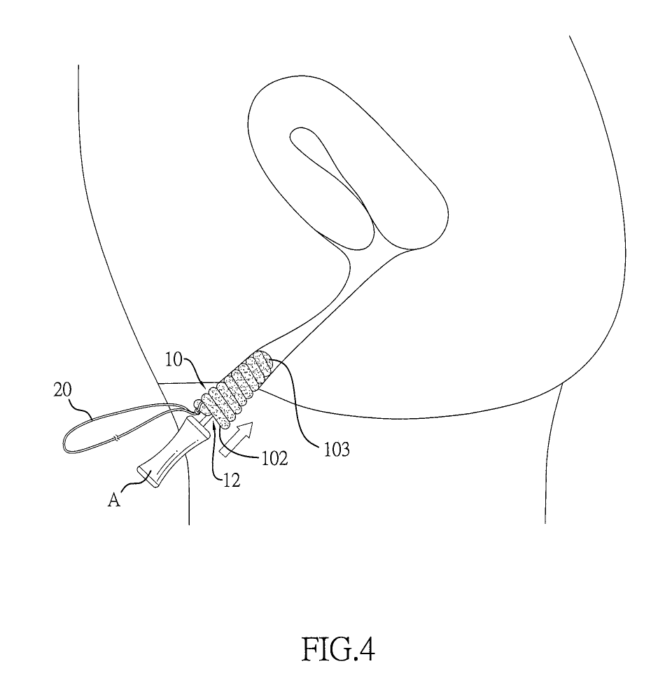

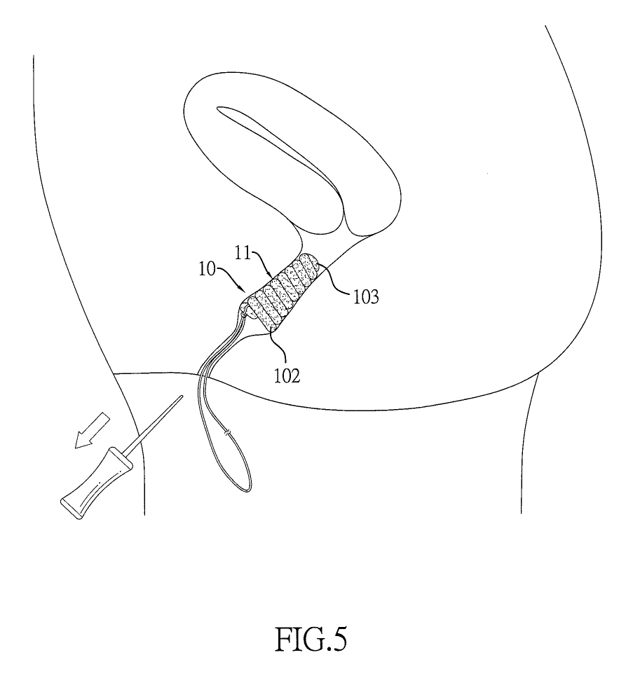

When the tampon is in use, referring to FIGS. 4 to 7, the smaller-diameter end 103 of the absorbent body 10 is first placed in the vaginal orifice, and then the tampon is pushed into the correct position at an upward angle of 45 degrees or by the applicator A, while ensuring that the string 20 is retained out of the vagina as shown in FIG. 5. Since the absorbent body 10 has the cushioning space 12, it has an air-permeation effect to provide better comfort.

Moreover, the absorbent body 10 has the larger-diameter end 102 and the smaller-diameter end 103, the smaller-diameter end 103 allows the user to push the absorbent body 10 into the vagina and place it in the correct position, the larger-diameter end 102 may be more closely abutted against the vaginal wall to prevent unabsorbed menstrual blood leakage, and the absorbent body 10 can be prevented from falling out due to improper placement.

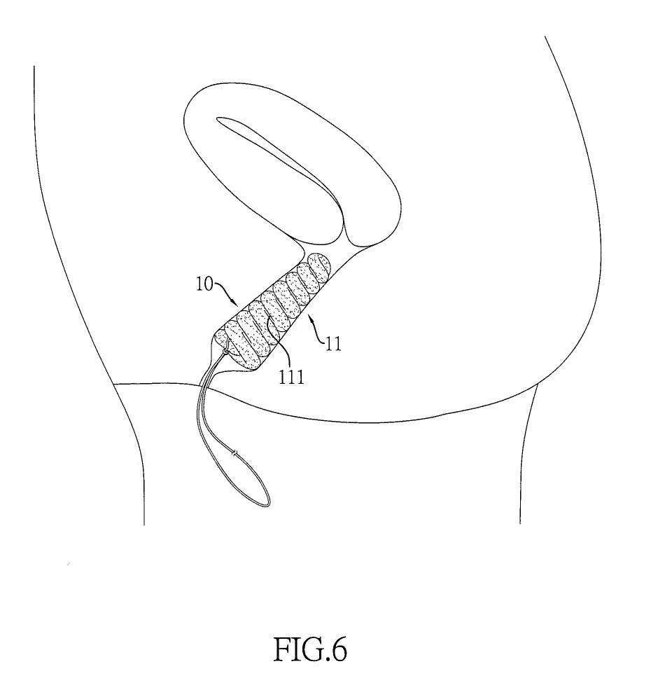

After a period of use, the absorbent body 10 absorbs menstrual blood and expands as shown in FIG. 6. The helical portion 11 of the absorbent body 10 is swollen and the previously tightly fitted multiple helical rings 111 are slightly loosened, so that the absorbent body 10 has a greater absorption surface area to absorb menstrual blood more quickly.

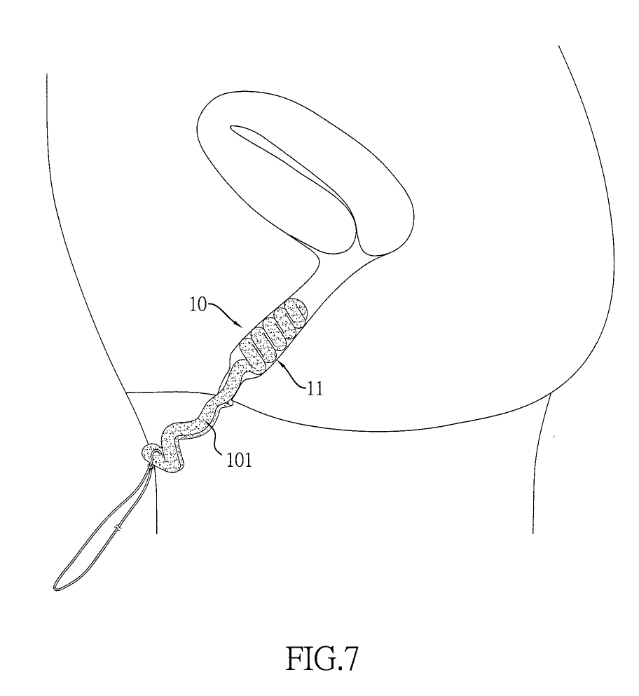

When the absorbent body 10 is to be removed, the helical portion 11 can be deformed by stretching the string 20 in the axial direction as shown in FIG. 7. The absorbent fiber 101 would become linear in shape due to the pulling, so that the absorbent body 10 may easily pass through the vaginal orifice and be removed without causing the user's discomfort due to the expansion of the absorbent body 10, and does not injure the user or cause inconvenience.

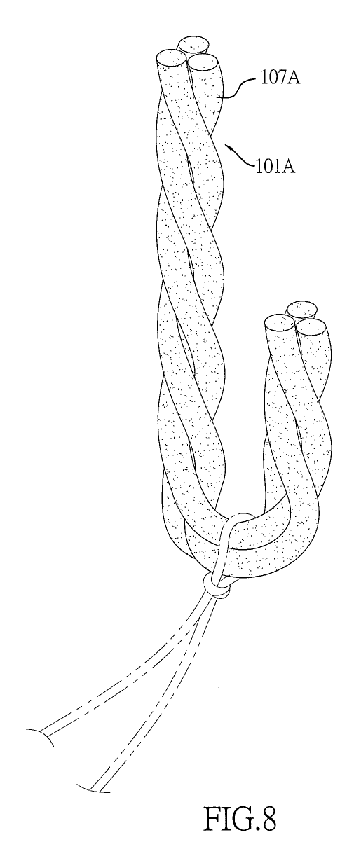

Referring to FIG. 8, the second embodiment of the present invention is similar to the first embodiment, but the difference is that the absorbent fiber 101A is formed by three fiber monomers 107A entwined to each other, then folded at a quarter of the full length to form a folding end and twined to form the tampon as in the first embodiment.

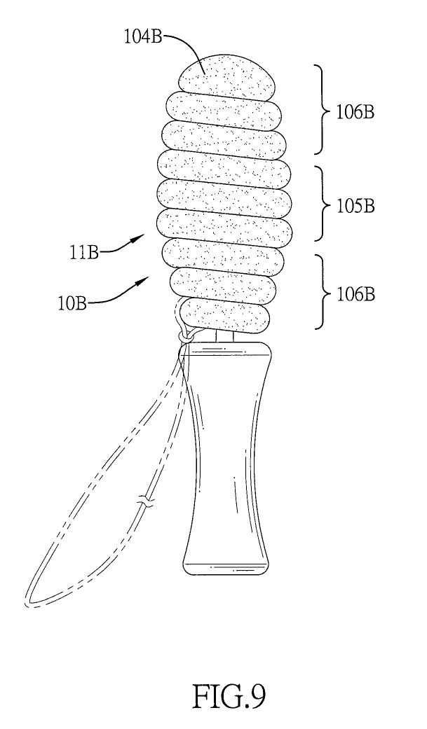

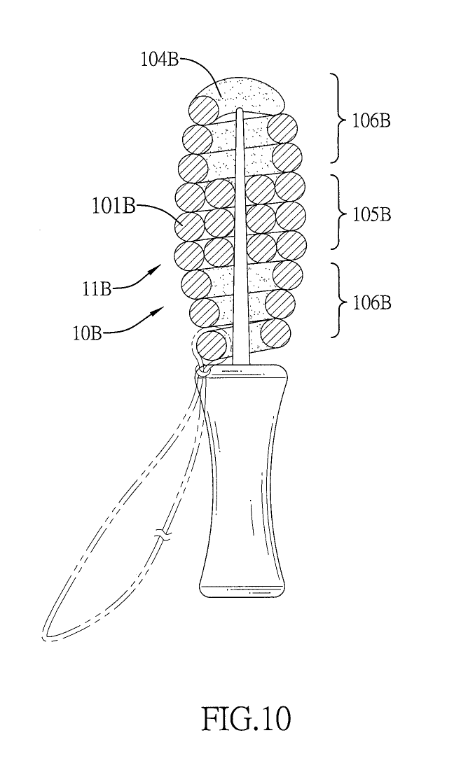

Referring to FIGS. 9 and 10, the third embodiment of the present invention is similar to the first embodiment, the difference is that the absorbent body 10B has a double thread segment 105B and two single thread segments 106B interconnected, and the double thread segment 105B is disposed between the two single thread segments 106B.

In the third embodiment of the present invention, the absorbent body 10 does not need to be folded to form a folding end, but is directly twined from the end opposite the shorter-diameter part 104B of the absorbent body 10, and the twining length is a quarter of the full length of the absorbent fiber 101B, thereby forming one of the two single thread segments 106B because of single-line twining. Then the absorbent fiber 101B is continuously twined to the length of half of the absorbent fiber 101B and twined at the same region again to form the double thread segment 105B; that is, the double thread segment 105B has a concentric double helix. Finally, the absorbent fiber 101B is twined to form the other two single thread segments 106B to complete the helical portion 11B of the absorbent body 10B.

According to the way to form the helical portion 11B of the absorbent fiber 101B, the absorbent body 10B of the third embodiment has the same characteristics and efficacy as those of the first embodiment except that the appearance of the absorbent body 10B of the third embodiment has two narrow ends and a width region between the two narrow ends.

To sum up, the tampon of the present invention provides improved comfort and ease of use. With the arrangement of the helical portion 11 and the cushion space 12, swelling of the absorbent body 10 may be mitigated to overcome the problems of discomfort caused by the conventional tampon, and the difficulty of withdrawing the tampon is reduced, and the tampon has better air permeability. In addition, as the absorbent body 10 has the larger-diameter end 102 and the smaller-diameter end 103 opposite each other, the tampon is easier to use and has the function of preventing blood leakage and preventing the tampon from falling off.

Even though numerous characteristics and advantages of the present invention have been set forth in the foregoing description, together with details of the structure and features of the invention, the disclosure is illustrative only. Changes may be made in the details, especially in matters of shape, size, and arrangement of parts within the principles of the invention to the full extent indicated by the broad general meaning of the terms in which the appended claims are expressed.

What is claimed is:

1. A tampon comprising:
an absorbent body, which has a helical portion and a cushion space, the cushion space axially formed in the helical portion; and
a string disposed on one end of the absorbent body.

2. The tampon according to claim 1, wherein the helical portion has multiple helical rings and each two adjacent ones of the multiple helical rings are closely abutted on each other.

3. The tampon according to claim 2, wherein the absorbent body has a double thread segment and a single thread segment interconnected, the double thread segment has a concentric double helix, and the single thread segment has a single helix.

4. The tampon according to claim 2, wherein the absorbent body has a double thread segment and two single thread segments interconnected, the double thread segment is disposed between the two single thread segments, the double thread segment has a concentric double helix, and the two single thread segments each have a single helix.

5. The tampon according to claim 1, wherein the absorbent body is formed by an absorbent fiber in twine, the absorbent fiber is twined to form the helical portion.

6. The tampon according to claim 5, wherein the absorbent fiber has a shorter-diameter part, which is adjacent to the end of the helical portion that is opposite the string.

7. The tampon according to claim 5, wherein the absorbent fiber is formed by multiple fiber monomers entwined.

8. The tampon according to claim 5, wherein the cross section of the absorbent fiber is circular.

9. The tampon according to claim 1, wherein both ends of the string are disposed on the absorbent body.

10. The tampon according to claim 1, wherein the absorbent body is cylindrical in shape, the absorbent body has a larger-diameter end and a smaller-diameter end opposite each other, and the string is disposed on the larger-diameter end.

11. The tampon according to claim 2, wherein the absorbent body is cylindrical in shape, the absorbent body has a larger-diameter end and a smaller-diameter end opposite each other, and the string is disposed on the larger-diameter end.

12. The tampon according to claim 3, wherein the absorbent body is cylindrical in shape, the absorbent body has a larger-diameter end and a smaller-diameter end opposite each other, and the string is disposed on the larger-diameter end.

13. The tampon according to claim 4, wherein the absorbent body is cylindrical in shape, the absorbent body has a larger-diameter end and a smaller-diameter end opposite each other, and the string is disposed on the larger-diameter end.

14. The tampon according to claim 5, wherein the absorbent body is cylindrical in shape, the absorbent body has a larger-diameter end and a smaller-diameter end opposite each other, and the string is disposed on the larger-diameter end.

15. The tampon according to claim 6, wherein the absorbent body is cylindrical in shape, the absorbent body has a larger-diameter end and a smaller-diameter end opposite each other, and the string is disposed on the larger-diameter end.

16. The tampon according to claim 7, wherein the absorbent body is cylindrical in shape, the absorbent body has a larger-diameter end and a smaller-diameter end opposite each other, and the string is disposed on the larger-diameter end.

17. The tampon according to claim 8, wherein the absorbent body is cylindrical in shape, the absorbent body has a larger-diameter end and a smaller-diameter end opposite each other, and the string is disposed on the larger-diameter end.
